(19) United States
(12) Patent Application Publication
(10) Pub. No.: US 2023/0058378 A1
(43) Pub. Date: Feb. 23, 2023

(54) ANTI-ITCH SCAR MANAGEMENT PRODUCTS, PROCESS OF MANUFACTURE AND USEFUL ARTICLES THEREOF

(71) Applicant: Mark E. Dillon, Chester Valley, PA (US)

(72) Inventor: Mark E. Dillon, Chester Valley, PA (US)

(73) Assignee: Bio Med Sciences, Inc., Allentown, PA (US)

(21) Appl. No.: 17/847,073

(22) Filed: Jun. 22, 2022

Related U.S. Application Data
(60) Provisional application No. 63/213,648, filed on Jun. 22, 2021.

(51) Int. Cl. A61L 26/00; B32B 5/02; B32B 27/12; B32B 5/18; B32B 37/24; B32B 27/06

(52) U.S. Cl. CPC A61L 26/0066; A61L 26/0019; B32B 5/028; B32B 27/12; B32B 5/18; B32B 5/022; B32B 37/24; B32B 27/065; A61L 2300/204; A61L 2300/216; A61L 2300/40; B32B 2260/02; B32B 2260/046; B32B 2437/00; B32B 2535/00; B32B 2305/18; B32B 2305/022; B32B 2305/07

(57) ABSTRACT

This invention relates to novel scar management products and methods of reducing dermal scars resulting from various types of dermal injuries while simultaneously reducing pruritis. Particularly, this invention relates to scar management products, each of which incorporate an antipruritic agent, that reduce and/or prevent dermal scarring and pruritis.

### CROSS REFERENCE TO RELATED APPLICATION

This application claims the benefit of U.S. Provisional Patent Application Ser. No. 63/213,648, which was filed on Jun. 22, 2021 and which is incorporated herein by reference in its entirety.

### BACKGROUND OF THE INVENTION

### 1. Field of the Invention

This invention relates to novel scar management products. Particularly, this invention relates to scar management products which incorporate an antipruritic agent. The product is applied to the scar area primarily to reduce and prevent dermal scaring, and, with the release of the antipruritic agent over time, to reduce pruritis (“itching”) during the scar maturation process. This invention is useful for pruritis mitigation for conditions unrelated to scarring as well.

### 2. Description of the Prior Art

It is well known that silicone-based sheeting and gel products are effective for the prevention or reduction (together or separately “management”) of dermal scaring. Pruritis is a common effect associated with wound healing and scar formation.

The effect is often particularly pronounced with wide-area wounds such as burns and abrasions, but also narrow wounds such as incisions and lacerations.

There are numerous scar management products available in the marketplace. Bio Med Sciences, Inc. of Allentown, Pa., manufactures and markets the Oleeva® and Silon® brands of scar management products including self-adhesive patches and dressings (for the purposes hereof, the term patch or dressing may be used interchangeably). My U.S. Pat. Nos. 4,832,009, 5,656,279, 5,980,923 and 7,087,135, which are incorporated herein by reference, describe wound dressings and scar management products utilizing an interpenetrating polymer network (“IPN”) of silicone and polytetrafluoroethylene. Bio Med Sciences also manufactures and markets other scar management and skin care products under various brands and in a variety of formats, including silicone-lined thermoplastic splinting as described in my U.S. Pat. No. 5,759,560, silicone-lined textiles as described in my U.S. Pat. No. 8,084,051, as well as silicone liquids and semisolid silicone paste applicator sticks. My U.S. Pat. No. 5,759,560 and my U.S. Pat. No. 8,084,051 also are incorporated herein by reference.

There are many common antipruritic agents commercially available, including antihistamines such as diphenhydramine and hydroxyzine, that block the effects of histamine which causes itching. Corticosteroids such as hydrocortisone cream and other topical steroids are likewise commonly used. Steroids work by activating natural substances in the skin to reduce swelling, redness, and itching. Counterirritants, such as mint oil, menthol, or camphor, local anesthetics such as lidocaine, pramoxine, or benzocaine are used as antipruritic agents in topical creams or lotions as well.

Diphenhydramine, commonly known as Benadryl®, is of particular interest. It has a long history of safe and effective topical application to temporarily relieve itching caused by minor burns/cuts/scrapes, sunburn, insect bites, minor skin irritations, or rashes from poison ivy, poison oak, or poison sumac.

Bio Med Sciences specifically contraindicates the use of creams or lotions under its silicone scar sheeting products because these sorts of substances can chemically degrade silicone thereby reducing the product's durability. In the case of self-adhesive dressings and patches, creams and lotions can also foul the skin-contacting surface of the scar sheets impacting their adhesive qualities and negatively affecting their clinical usefulness.

A Franz Cell chamber is an in vitro skin permeation assay frequently used in transdermal drug delivery system analysis. The apparatus consists of two primary chambers separated by a membrane, such as Strat-M membrane from Millipore Sigma company of Burlington, Mass., USA. The test product is applied to the membrane via the top chamber. The bottom chamber contains fluid from which samples are taken at regular intervals for analysis to determine the release kinetics of active pharmaceutical ingredients in the test product through the membrane. The chamber is typically maintained at a constant temperature of 37° C. to mimic human clinical usage.

### SUMMARY OF THE INVENTION

In an effort to improve the art, I have created a scar management product containing an antipruritic agent to alleviate pruritis while not diminishing the adhesive and clinical qualities of the scar patch. This is particularly useful because the combined application of silicone scar products with antipruritic creams or lotions is problematic. The alternative of removing the scar management product to apply creams or lotions periodically for continuity of the clinical effect is further problematic from a practicality and compliance perspective. The present invention obviates the problematic issues of intermittent use of creams or lotions in combination with silicone scar management patches while providing prolonged delivery of an antipruritic agent for localized antipruritic effect.

### BRIEF DESCRIPTION OF THE DRAWINGS

FIG. 1 shows schematic representation of a partial view in cross-section of a preferred scar management dressing constructed in accordance with the invention, in which reference number 100 refers to a monolithic design with an antipruritic agent homogeneously dispersed in the silicone matrix 105. FIG. 2 shows a bilayer design that comprises the skin contacting surface 210 and an outer distal layer 220.

### DETAILED DESCRIPTION

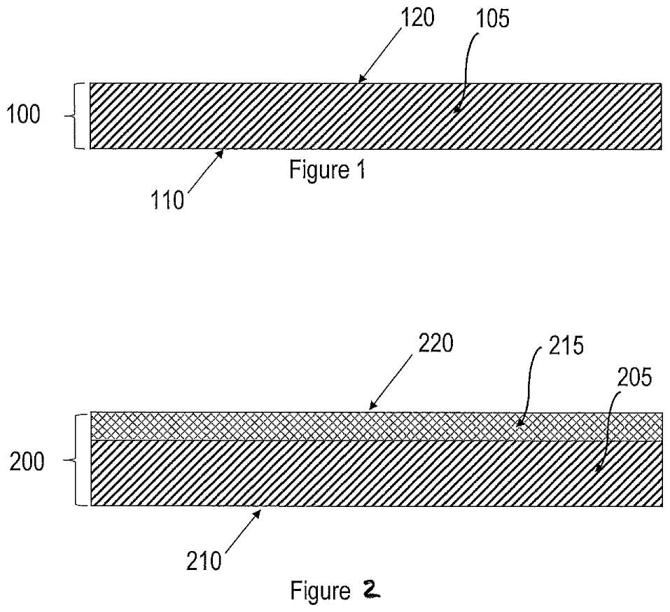

In a preferred embodiment, the invention, as illustrated in FIG. 1, comprises a dressing 100 having a silicone matrix 105 with an antipruritic agent homogeneously dispersed throughout the silicone matrix 105. The dressing 100 has a skin contacting surface 110 and an outer surface 120. In the preferred embodiment of the invention shown in FIG. 2, the dressing 200 has a layer comprised of silicone matrix 205 having an antipruritic agent homogeneously dispersed throughout the silicone matrix 205. The dressing 200 has a skin contacting side 210, a distal non-skin contacting backing layer 215, and an outer distal layer 220.

My inventive scar management dressing for preventing or reducing dermal scarring and reducing pruritic comprises a skin contacting layer having a skin contacting side, and an antipruritis agent homogeneously dispersed throughout the skin contacting layer.

Preferably, the skin contacting layer of the dressing comprises a silicone-based material, such as silicone, polydimethylsiloxane, polyorganosiloxane, silicone gel, or silicone elastomer. The dressing also may have additional layers positioned over the skin contacting layer, such as an interpenetrating polymer network (“IPN”), or a polymer that forms an interpenetrating polymer network (“IPN”) with the skin contacting layer, or an outer backing layer such as textile fabric, foam, non-woven film, mesh, thermoplastic material, or material other than textile fabric. The dressing may comprise Oleeva® scar management dressings, Silon® scar management dressings, or any of the dressings described in my U.S. Pat. Nos. 4,832,009, 5,656,279, 5,980,923, 7,087,135, 5,759,560, and 8,084,051, wherein all of such dressings have an antipruritic agent homogeneously dispersed throughout the skin contacting layer thereof.

Preferably, the antipruritic agent comprises one or more of the following: diphenylhydramine HCl, corticosteroids, hydrocortisone, other topical steroids, diphenhydramine, counterirritants, such as mint oil, menthol, or camphor, and local anesthetics, such as lidocaine, pramoxine, or benzocaine.

Preferably, the skin contacting layer comprises about 1% to about 20% by weight antipruritic agent, and more preferably comprises about 10% by weight antipruritic agent.

A number of methods may be used to manufacture my inventive scar management dressing for preventing or reducing scarring and reducing pruritis. In general, during the formation of the skin contacting layer of the dressing, an antipruritic agent is dispersed throughout the material (e.g., the silicone-based material) that forms the skin contacting layer of the dressing prior to the formation of the skin contacting layer. For instance, the methods of manufacturing my inventive scar management dressing for preventing or reducing dermal scarring and reducing pruritis may comprise any of the methods described in my U.S. Pat. Nos. 4,832,009, 5,656,279, 5,980,923, 7,087,135, 5,759,560, and 8,084,051, except that an antipruritic agent is dispersed throughout the material (e.g., the silicone-based material) that forms the skin contacting layer of the dressings disclosed in my U.S. Pat. Nos. 4,832,009, 5,656,279, 5,980,923, 7,087,135, 5,759,560, and 8,084,051, prior to the formation of the skin contacting layer.

In use, the scar management dressing of the invention is placed over a patient's wound, such that the skin contacting layer is in contact with the wound to prevent or reduce dermal scarring, while simultaneously providing prolonged delivery of an antipruritic agent from the skin contacting layer for localized antipruritic effect. The following examples are not intended to be limiting. The following examples illustrate various preferred embodiments of the invention.

### Baseline:

As a benchmark measurement to compare to over-the-counter cream, Extra Strength Benadryl® (2% diphenhydramine, Johnson and Johnson, New Brunswick, N.J.) was applied to the surface of an artificial skin equivalent, which in this baseline example and the Examples 1-4 set out below comprises the membrane, such as a Strat-M membrane from Millipore Sigma, separating the two primary chambers of a Franz Cell chamber. A Franz Cell chamber was used according to established methods and generated the following results:

Diphenhydramine 2% cream applied to artificial skin equivalent:

Baseline

| Time | Average release | |
|---|---|---|
| (hr) | (μg/cm²) | StDev |
| 0.5 | 2.1 | 1.6 |
| 1 | 3.5 | 1.3 |
| 3 | 5.5 | 1.2 |
| 6 | 6.1 | 1.0 |
| 24 | 7.4 | 2.3 |
| 48 | 7.2 | 0.4 |

### EXAMPLES 1-4

Four (4) Silon IPN films with textile backing layers were produced according to established methods, except that the antipruritic agent used in these Examples 1-4 was dispersed throughout the silicone phase of the IPN film. Two (2) silicone formulations were used at two (2) IPN coating thicknesses. The silicones were polydimethylsiloxanes, and for these examples were two-component, addition-cured compositions commercially sold as skin adhesives as Silpuran® 2130 and Silpuran® 2114 (Wacker Chemical Corp., Adrian, Mich., USA).

Each of the four Silon IPN films (that is, each of the specimens) contained 10% by weight diphenhydramine (LGM Pharma, Erlanger, Ky., USA) in the silicone phase of the IPN. All were produced under identical conditions and produced as follows:

| | TPN Coating Thickness | |
|---|---|---|
| Sample | Setting | Silicone formulation |
| 1 | 27 mil (686 micron) | Silpuran 2130 |
| 2 | 10 mil (254 micron) | Silpuran 2130 |
| 3 | 27 mil (686 micron) | Silpuran 2114 |
| 4 | 10 mil (254 micron) | Silpuran 2114 |

Each specimen was tested on a Franz Cell chamber using the artificial skin equivalent described above. The results for the baseline and four (4) example specimens were as follows:

| Base-Line | | | Sample 1 | | | Sample 2 | | | Sample 3 | | | Sample 4 | | |
|---|---|---|---|---|---|---|---|---|---|---|---|---|---|---|
| | Average | | | Average | | | Average | | | Average | | | Average | |
| Time | release | | Time | release | | Time | release | | Time | release | | Time | release | |
| (hr) | (μg/cm²) | StDev | (hr) | (μg/cm²) | StDev | (hr) | (μg/cm²) | StDev | (hr) | (μg/cm²) | StDev | (hr) | (μg/cm²) | StDev |
| 0.5 | 2.1 | 1.6 | 0.5 | 1.7 | 0.8 | 0.5 | 45.1 | 9.7 | 0.5 | 9.8 | 3.2 | 0.5 | 44.3 | 12.6 |
| 1 | 3.5 | 1.3 | 1 | 2.3 | 1.4 | 1 | 45.5 | 14.8 | 1 | 11.7 | 1.4 | 1 | 45.2 | 9.1 |
| 3 | 5.5 | 1.2 | 3 | 4.0 | 1.6 | 3 | 48.6 | 11.4 | 3 | 16.2 | 2.9 | 3 | 42.2 | 2.0 |
| 6 | 6.1 | 1.0 | 6 | 5.0 | 1.8 | 6 | 50.7 | 13.6 | 6 | 19.1 | 3.6 | 6 | 49.9 | 9.1 |
| 24 | 7.4 | 2.3 | 24 | 5.8 | 1.2 | 24 | 44.6 | 10.9 | 24 | 21.5 | 2.7 | 24 | 52.6 | 9.4 |
| 48 | 7.2 | 0.4 | 48 | 6.5 | 1.3 | 48 | 53.0 | 14.7 | 48 | 18.9 | 4.8 | 48 | 55.4 | 7.9 |

Additional examples illustrating my invention are as follows:

### EXAMPLE 5

A Silon IPN film with textile backing layer is produced according to established methods, except that the antipruritic agent used in this Example 5 is dispersed throughout the silicone phase of the IPN film. A two-component, addition-cured silicone composition commercially sold as MED-6350 (Avantor, Radnor, Pa. 19087) is used as the silicone phase of the IPN film, and is blended with diphenhydramine (LGM Pharma, Erlanger, Ky., USA) such that the silicone phase of the IPN film comprises 10% by weight diphenhydramine, and the IPN film is cast at a thickness of 27 mil (686 micron).

### EXAMPLE 6

A Silon IPN film with textile backing layer is produced according to established methods, except that the antipruritic agent used in this Example 6 is dispersed throughout the silicone phase of the IPN film. A two-component, addition-cured silicone composition commercially sold as MED-6350 (Avantor, Radnor, Pa. 19087) is used as the silicone phase of the IPN film, and is blended with diphenhydramine (LGM Pharma, Erlanger, Ky., USA) such that the silicone phase of the IPN film comprises 1% by weight diphenhydramine, and the IPN film is cast at a thickness of 27 mil (686 micron).

### EXAMPLE 7

A Silon IPN film with textile backing layer is produced according to established methods, except that the antipruritic agent used in this Example 7 is dispersed throughout the silicone phase of the IPN film. A two-component, addition-cured silicone composition commercially sold as MED-6350 (Avantor, Radnor, Pa. 19087) is used as the silicone phase of the IPN film, and is blended with diphenhydramine (LGM Pharma, Erlanger, Ky., USA) such that the silicone phase of the IPN film comprises 20% by weight diphenhydramine, and the IPN film is cast at a thickness of 27 mil (686 micron).

### EXAMPLE 8

A Silon IPN film with textile backing layer is produced according to established methods, except that the antipruritic agent used in this Example 8 is dispersed throughout the silicone phase of the IPN film. A two-component, addition-cured silicone composition commercially sold as MED-6350 (Avantor, Radnor, Pa. 19087) is used as the silicone phase of the IPN film, and is blended with hydrocortisone (Sigma-Aldrich, Purchasable Chemical: 1317007 USP, Saint Louis, Mo., USA) such that the silicone phase of the IPN film comprises 10% by weight hydrocortisone, and the IPN film is cast at a thickness of 27 mil (686 micron).

1. A scar management dressing for reducing and/or preventing dermal scarring and for alleviating pruritis, comprising
a skin contacting layer having a skin contacting side, and
an antipruritic agent dispersed throughout the skin contacting layer.

2. The scar management dressing of claim 1,
the skin contacting layer comprising 1% to 20% by weight antipruritic agent.

3. The scar management dressing of claim 1,
the skin contacting layer comprising 10% by weight antipruritic agent.

4. The scar management product of claim 1,
the skin contacting layer comprising a silicone-based material.

5. The scar management dressing of claim 4,
the silicone-based material comprising silicone, polydimethylsiloxane, polyorganosiloxane, silicone gel, or a silicone elastomer.

6. The scar management dressing of claim 1,
the antipruritic agent comprising diphenylhydramine HCl, corticosteroids, hydrocortisone, diphenhydramine, mint oil, menthol, camphor, lidocaine, pramoxine, and/or benzocaine.

7. The scar management dressing of claim 1,
the antipruritic agent comprising topical steroids, counterirritants, and/or local anesthetics.

8. The scar management dressing of claim 1, further including
a remainder portion of the scar management dressing positioned on the skin contacting layer,
the remainder portion including an interpenetrating polymer network, a polymer that forms an interpenetrating polymer network with the skin contacting layer, textile fabric, foam, non-woven film, mesh, thermoplastic material, and/or a material other than a textile fabric.

9. A method of manufacturing a scar management dressing, comprising the steps of
mixing an antipruritic agent into a silicone formulation to form a mixture thereof,
the silicone formulation comprising polyorganosiloxane, silicone, silicone gel, silicone elastomer, or polydimethylsiloxane,
applying the mixture onto a carrier to form a layer of the mixture thereon, and
curing the layer of the mixture.

10. The method of claim 9,
the antipruritic agent comprising diphenylhydramine HCl, corticosteroids, hydrocortisone, diphenhydramine, mint oil, menthol, camphor, lidocaine, pramoxine, and/or benzocaine.

11. The method of claim 9,
the antipruritic agent comprising topical steroids, counterirritants, and/or local anesthetics.

12. The method of claim 9, further including
applying expanded polytetrafluoroethylene to the layer of the mixture prior to curing.

13. The method of claim 12,
the silicone formulation being silicone,
the layer of the mixture and the expanded polytetrafluoroethylene forming an interpenetrating polymer network of silicone and polytetrafluoroethylene, the interpenetrating polymer network having a first side and a second side, the first side forming the skin contacting side of the skin contacting layer, and further including the step of
laminating thermoplastic splinting material to the second side of the interpenetrating polymer network.

14. The method of claim 9, further including
applying an apertured mesh layer to the layer of the mixture prior to curing.

15. The method of claim 9,
further including, prior to curing, the steps of
applying a microporous polymer sheeting membrane to the layer of the mixture and allowing or causing the layer of the mixture to impregnate the microporous polymer sheeting membrane, and
applying a backing material to the distal surface of the impregnated microporous polymer sheeting material, with the backing material being in contact with the mixture that has made its way through the microporous polymer sheeting membrane.

16. The method of claim 15,
the backing material comprising textile fabric, foam, non-woven film, or material other than a textile fabric.

17. The method of claim 9, further including, prior to curing, the steps of
applying a lamination of a microporous polymer sheeting membrane and backing material to the layer of the mixture and allowing or causing the layer of the mixture to impregnate the microporous polymer sheeting membrane.

18. The method of claim 17,
the backing material comprising textile fabric, foam, non-woven film, or material other than a textile fabric.

19. A method of manufacturing a scar management dressing, comprising the steps of
mixing an antipruritic agent into a silicone formulation to form a mixture thereof,
applying the mixture onto a textile fabric to form a textile fabric having the mixture applied thereto, and
fabricating the textile fabric having the mixture applied thereto into a scar management dressing.

20. The method of claim 19,
the scar management dressing being a garment.

21. A method of reducing dermal scars, comprising the steps of
providing the scar management dressing of claim 1,
applying the skin contacting side of the skin contacting layer of the scar management dressing over and into contact with a closed wound site, and
maintaining the scar management dressing over and into contact with the closed wound site for an effective amount of time to reduce and/or prevent dermal scarring and to alleviate pruritis.
